(19) United States
(12) Patent Application Publication
(10) Pub. No.: US 2024/0148546 A1
(43) Pub. Date: May 9, 2024

(54) SYSTEM FOR THERMAL AND COMPRESSION THERAPY FOR REDUCTION OF ALOPECIA IN CHEMOTHERAPY PATIENTS AND METHOD OF USE

(71) Applicant: Hair Science Systems LLC, Cincinnati, OH (US)

(72) Inventor: William Banker, Cincinnati, OH (US)

(73) Assignee: Hair Science Systems LLC, Cincinnati, OH (US)

(21) Appl. No.: 18/500,189

(22) Filed: Nov. 2, 2023

Related U.S. Application Data
(60) Provisional application No. 63/382,160, filed on Nov. 3, 2022.

(51) Int. Cl. A61F 7/02; A61H 9/00

(52) U.S. Cl. CPC A61F 7/02; A61H 9/0078; A61F 2007/0008; A61H 2201/0214; A61H 2205/021; A61H 2209/00

(57) ABSTRACT

A system includes a treatment cap assembly and a cooling and compression unit coupled to the treatment cap assembly. The treatment cap assembly comprises a fluid circulating pad configured to circulate a cooling fluid; and a compression hood configured to administer compression to a scalp of an individual wearing the treatment cap assembly. The cooling and compression unit is configured to provide the cooling fluid to the fluid circulating pad and to periodically provide compressed gas to the compression hood. The system can be used in a method that includes administering to an individual a dosage of a chemotherapeutic agent; cooling a scalp of the individual to a temperature of less than about 65° F.; and periodically administering compression to the scalp of the individual during the step of administering the dosage of the chemotherapeutic agent.

### CROSS-REFERENCE TO RELATED APPLICATIONS

This application claims priority to and all benefit of U.S. Provisional Patent Application No. 63/382,160, filed on Nov. 3, 2022, the entire disclosure of which is fully incorporated herein by reference.

### BACKGROUND

Chemotherapy frequently induces hair loss (also referred to as alopecia), which can be a traumatic experience for some patients, and may result in lower self-esteem and increased resistance to chemotherapy treatments by patients as a result of a fear of lo sing their hair.

Scalp tourniquets have been used for decades in an attempt to prevent chemotherapy-induced alopecia. This technique involves placement of a pneumatic tourniquet around the hairline during administration of the chemotherapy drugs. The tourniquet is then inflated to a pressure above the systolic arterial pressure in order to reduce blood flow to the scalp. However, the effectiveness of this technique has not been unambiguously demonstrated.

More recently, a technique known as scalp hypothermia has been replacing the use of scalp tourniquets. With scalp hypothermia, the scalp temperature is lowered to below 24° C. (75° F.) through the application of cold packs or the like prior to administration of the chemotherapy drugs. Although this technique has been reported to afford a 50-70% reduction in the amount of hair loss, the results are notoriously variable. Additionally, the use of fluid circulating caps to reduce the scalp temperature to such a low temperature can be uncomfortable for patients and does not prevent damage to the hair follicles by the chemotherapy drugs.

Accordingly, there remains a need for alternative methods of reducing alopecia in chemotherapy patients.

### SUMMARY

Various exemplary aspects of the inventive concepts are directed to a prophylactic treatment for alopecia comprising: administering to an individual a dosage of a chemotherapeutic agent; cooling a scalp of the individual to a temperature of less than about 65° F.; and periodically administering compression to the scalp of the individual during the step of administering the dosage of the chemotherapeutic agent.

According to various aspects, a method comprises administering to an individual a dosage of a chemotherapeutic agent; cooling a scalp of the individual to a temperature of from about 58° F. to about 65° F. (from about 14.4° C. to about 18.3° C.); and periodically administering compression to the scalp of the individual during the step of administering the dosage of the chemotherapeutic agent.

In some aspects, a method comprises the method of any of the previous aspects, wherein periodically administering compression to the scalp comprises: administering compression for a time period of from about 5 minutes to about 20 minutes; a period of rest of from about 2 minutes to about 10 minutes during which compression is not administered; and repeating the administering compression and the period of rest.

In some aspects, a method comprises the method of any of the previous aspects, wherein the cooling the scalp begins prior to the administering to the individual the dosage of the chemotherapeutic agent.

In some aspects, a method comprises the method of any of the previous aspects, wherein the cooling the scalp continues following completion of the administering to the individual the dosage of the chemotherapeutic agent.

In some aspects, a method comprises the method of any of the previous aspects, wherein the periodically administering compression begins prior to the administering to the individual the dosage of the chemotherapeutic agent.

In some aspects, a method comprises the method of any of the previous aspects, wherein the periodically administering compression continues following completion of the administering to the individual the dosage of the chemotherapeutic agent.

According to some aspects provided herein, a system comprises a treatment cap assembly and a cooling and compression unit coupled to the treatment cap assembly. The treatment cap assembly comprises a fluid circulating pad configured to circulate a cooling fluid, and a compression hood configured to administer compression to a scalp of an individual wearing the treatment cap assembly. The cooling and compression unit is configured to provide the cooling fluid to the fluid circulating pad and to periodically provide compressed gas to the compression hood.

In some aspects, the system comprises the system of the previous aspect, wherein the fluid circulating pad comprises: one or more fluid channels through which the cooling fluid is circulated; a fluid inlet in fluid communication with the cooling and compression unit to receive cooling fluid from the cooling and compression unit; and a fluid outlet in fluid communication with the cooling and compression unit to provide the cooling fluid to the cooling and compression unit after circulation through the one or more fluid channels.

In some aspects, the system comprises the system of any of the previous aspects, wherein the fluid circulating pad includes one or more sensors communicatively coupled to the cooling and compression unit and configured to collect and transmit information to the cooling and compression unit.

In some aspects, the system comprises the system of any of the previous aspects, wherein the compression hood is removably coupled to the fluid circulating pad.

In some aspects, the system comprises the system of any of the previous aspects, wherein the compression hood comprises: an inlet conduit fluidly coupled to the cooling and compression unit to receive compressed gas from the cooling and compression unit; and a vent configured to controllably release air from the compression hood.

In some aspects, the system comprises the system of any of the previous aspects, wherein the cooling and compression unit is configured to: provide compressed gas to the compression hood for a time period of from about 5 minutes to about 20 minutes; cease providing compressed gas to the compression hood for a period of rest of from about 2 minutes to about 10 minutes; and repeat the providing compressed gas and the period of rest.

In another aspect, a system or method includes the system or method according to any previous aspect, wherein the time period for providing compressed gas to the compression hood is from about 7.5 minutes to about 18 minutes.

In another aspect, a system or method includes the system or method according to any previous aspect, wherein the time period for providing compressed gas to the compression hood is from about 10 minutes to about 15 minutes.

In another aspect, a system or method includes the system or method according to any previous aspect, wherein the period of rest is from about 3 minutes to about 7 minutes.

In another aspect, a system or method includes the system or method according to any previous aspect, wherein the period of rest is from about 4 minutes to about 7 minutes.

In another aspect, a system or method includes the system or method according to any previous aspect, wherein the compression is administered at a pressure of from about 0.5 mmHg to about 33 mmHg above ambient pressure.

In another aspect, a system or method includes the system or method according to any previous aspect, wherein the circulating cooling fluid is effective to cool the scalp of the individual to a temperature of from about 35° F. to about 65° F.

In another aspect, a system or method includes the system or method according to any previous aspect, wherein the circulating cooling fluid is effective to cool the scalp of the individual to a temperature of from about 60° F. to about 62° F.

In another aspect, a system or method includes the system or method according to any previous aspect, wherein administering compression to the scalp of the individual is effective to reduce blood flow to follicles of the scalp from about 10% to about 50% relative to an amount of blood flow to the follicles observed when compression is not administered to the scalp.

In another aspect, a system or method includes the system or method according to any previous aspect, wherein administering compression to the scalp of the individual is effective to reduce blood flow to follicles of the scalp from about 20% to about 40% relative to an amount of blood flow to the follicles observed when compression is not administered to the scalp.

Numerous other aspects, advantages, and/or features of the general inventive concepts will become more readily apparent from the following detailed description of exemplary embodiments and from the accompanying drawings being submitted herewith.

### BRIEF DESCRIPTION OF THE DRAWINGS

The general inventive concepts, as well as illustrative embodiments and advantages thereof, are described below in greater detail, by way of example, with reference to the drawings in which:

FIG. 1 illustrates an example treatment cap assembly fully fitted on a patient in accordance with one or more embodiments shown and described herein;

FIG. 2 illustrates an example circulating pad in accordance with one or more embodiments shown and described herein;

FIG. 3 illustrates another example treatment cap assembly in accordance with one or more embodiments shown and described herein;

FIG. 4 illustrates the example treatment cap assembly of FIG. 3 fully fitted on a patient in accordance with one or more embodiments shown and described herein;

FIG. 5 is a cross-sectional view of a circulating pad in accordance with one or more embodiments shown and described herein;

FIG. 6 is another example of a treatment cap assembly in accordance with one or more embodiments shown and described herein;

FIG. 7 is a top view of the inner layer of the treatment cap assembly of FIG. 6;

FIG. 8 is a cross-sectional view of the treatment cap assembly of FIGS. 6 and 7;

FIG. 9 illustrates the example cold treatment assembly of FIG. 6 fully fitted on a patient in accordance with one or more embodiments shown and described herein;

FIG. 10 is a perspective view of a cooling and compression unit for thermal and compression therapy relative to the prevention of alopecia in chemotherapy patients in accordance with one or more embodiments shown and described herein;

FIG. 11 is a cut-away, perspective view of the cooling and compression unit of FIG. 10 in accordance with one or more embodiments shown and described herein;

FIG. 12 is a cut-away, perspective view of the cooling and compression unit of FIG. 10 taken from the opposite side of that of FIG. 11;

FIG. 13 is a rear perspective view of the cooling and compression unit of FIG. 10 in accordance with one or more embodiments shown and described herein;

FIG. 14 is a diagrammatic schematic of a cooling and compression unit and treatment assembly cap system illustrating the integration of thermal and compression elements in accordance with one or more embodiments shown and described herein;

FIG. 15 is an illustration of a treatment cap assembly connected to a cooling and compression unit in accordance with one or more embodiments shown and described herein;

FIG. 16A is a diagrammatic schematic of an aspect of thermal operation of the cooling and compression unit according to one or more embodiments shown and described herein;

FIG. 16B is a rear view of an example integrated reservoir and heat transfer assembly in accordance with one or more embodiments shown and described herein;

FIG. 16C is a perspective view of an example integrated reservoir and heat transfer assembly in accordance with one or more embodiments shown and described herein;

FIG. 17 is a cooling and compression system block diagram in accordance with one or more embodiments shown and described herein;

FIG. 18 is a block diagram illustrating operation of a cooling and compression system in accordance with one or more embodiments shown and described herein; and

FIG. 19 is a cooling and compression system block diagram in accordance with one or more embodiments shown and described herein.

### DETAILED DESCRIPTION

Unless otherwise defined, all technical and scientific terms used herein have the same meaning as commonly understood by one of ordinary skill in the art to which these exemplary embodiments belong. The terminology used in the description herein is for describing exemplary embodiments only and is not intended to be limiting of the exemplary embodiments. Accordingly, the general inventive concepts are not intended to be limited to the specific embodiments illustrated herein. Although other methods and materials similar or equivalent to those described herein can be used in the practice or testing of the present invention, the preferred methods and materials are described herein.

As used in the specification and the appended claims, the singular forms “a,” “an,” and “the” are intended to include the plural forms as well, unless the context clearly indicates otherwise.

Unless otherwise indicated, all numbers expressing quantities of ingredients, chemical and molecular properties, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present exemplary embodiments. At the very least, each numerical parameter should be construed in light of the number of significant digits and ordinary rounding approaches.

Unless otherwise indicated, any element, property, feature, or combination of elements, properties, and features, may be used in any embodiment disclosed herein, regardless of whether the element, property, feature, or combination of elements, properties, and features was explicitly disclosed in the embodiment. It will be readily understood that features described in relation to any particular aspect described herein may be applicable to other aspects described herein provided the features are compatible with that aspect. In particular: features described herein in relation to the method may be applicable to the system and vice versa.

Every numerical range given throughout this specification and claims will include every narrower numerical range that falls within such broader numerical range, as if such narrower numerical ranges were all expressly written herein.

The general inventive concepts relate to a system and method for reducing or even preventing chemotherapy-induced alopecia. In various embodiments described herein, scalp cooling is used in combination with application of dynamic pressure during administration of chemotherapy drugs to reduce the exposure of hair follicles to alopecia-inducing products of chemotherapy. Surprisingly, it has been found that the application of dynamic pressure can enable the less cooling to be applied to the scalp while realizing the same or improved levels of hair loss as compared to application of cooling alone or in combination with static pressure. The decreased amount of cooling can make the treatment more comfortable for patients.

In some embodiments, scalp cooling and compression is commenced prior to the administration of the chemotherapeutic agent(s) to the patient such that the scalp is sufficiently cooled when administration of the agents begins and blood flow to the scalp is reduced as compared to a typical blood flow to the scalp for the patient. In various embodiments, scalp cooling and/or compression can be continued during and after administration of the chemotherapeutic agents has been completed, as will be described in greater detail below.

In one exemplary treatment regime, scalp cooling begins prior to administration of the chemotherapeutic agents. For example, the scalp cooling can begin about 30 minutes prior to the administration of the chemotherapeutic agents. In embodiments, scalp compression can begin at the same time that scalp cooling begins, between the beginning of scalp cooling and the beginning of administration of the chemotherapeutic agents, or at the same time that the administration of the chemotherapeutic agents begins. For example, scalp cooling and compression can begin about 30 minutes prior to the administration of the chemotherapeutic agents, scalp cooling can begin about 30 minutes prior to the administration of the chemotherapeutic agents and scalp compression can begin about 15 minutes prior to the administration of the chemotherapeutic agents, or scalp cooling can begin about 30 minutes prior to the administration of the chemotherapeutic agents and scalp compression can begin at the same time that administration of the chemotherapeutic agents begins.

In various embodiments, scalp cooling and scalp compression are continued during administration of the chemotherapeutic agents (e.g., during infusion). As will be described herein, scalp compression can be administered dynamically such that the amount of compression can change as a function of time during administration of the chemotherapeutic agents. For example, scalp compression can be administered for a period of about 15 minutes, followed by a period of about 5 minutes during which scalp compression is not administered, followed by a period of about 15 minutes during which scalp compression is administered (e.g., 15 minutes on, 5 minutes off). Accordingly, it should be understood that the compression level can change during the administration of the chemotherapeutic agents.

Since the chemotherapeutic agents will continue to circulate in the bloodstream for a period of time following the end of the administration, continuing scalp cooling and compression beyond the conclusion of administration of the chemotherapeutic agents can continue to protect hair follicles. Accordingly, in embodiments, scalp cooling and compression are continued for a period of time following the completion of the administration of the chemotherapeutic agents. For example, scalp cooling and compression can be continued for about 1 hour, for about 2 hours, for about 3 hours, for about 4 hours, or for about 5 hours after administration of the chemotherapeutic agent has ended. However, it is contemplated that the period of cooling and compression following the completion of the administration of the chemotherapeutic agents can be adjusted based on, for example, the half-life to the chemotherapeutic agents administered to the patient, the comfort of the patient, and other factors.

In various embodiments, a treatment cap assembly (sometimes referred to herein as a “treatment cap”) is used in combination with a cooling and compression unit. As will be described, the cooling and compression unit enables the rate of cooling of the scalp and level of compression to be controlled which can lead to improved tolerance of the cooling by the patient. Various examples of treatment cap assemblies are described herein.

### Treatment Cap Assemblies

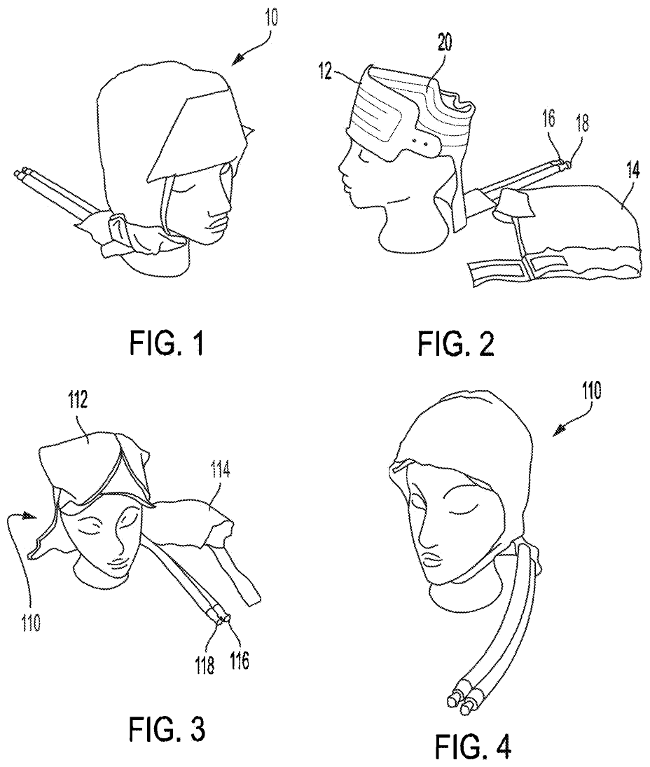

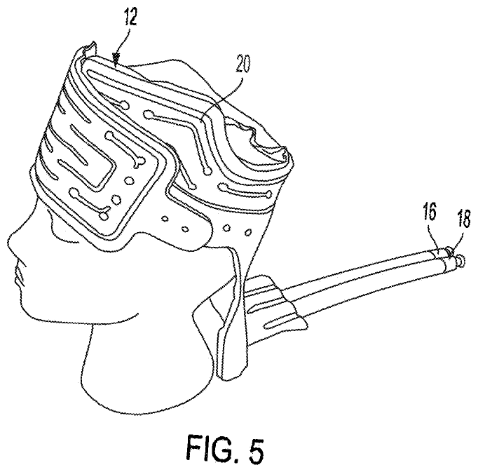

In various embodiments, the treatment cap assembly is configured to extend around the patient's scalp. The treatment cap assembly generally includes one or more fluid channels through which a cooling fluid (e.g., water) is circulated. The one or more fluid channels are arranged such that the cooling fluid circulated therethrough will cool the patient's scalp, absorbing heat from the patient's scalp, and transporting it away from the scalp. In FIGS. 1, 2, and 5, the treatment cap assembly 10 includes a fluid circulating pad 12 configured to be wrapped around a patient's head. The fluid circulating pad 12 includes one or more fluid channels 20 formed therein, as well as a fluid inlet 16 and a fluid outlet 18. The fluid inlet 16 and the fluid outlet 18 are in fluid communication with one another through the one or more fluid channels 20 such that the cooling fluid enters the one or more fluid channels 20 through the fluid inlet 16 and flows through the one or more fluid channels 20 to the fluid outlet 18.

The fluid inlet 16 and the fluid outlet 18 can be configured for attachment to corresponding fluid inlet and fluid outlet ports on the cooling and compression unit 40. Any one of a number of connector elements can be employed to couple the fluid inlet 16 and the fluid outlet 18 to the ports of the cooling and compression unit 40. For example, the fluid inlet 16 and the fluid outlet 18 can be connected to the ports of the cooling and compression unit 40 through various tubes that are coupled between the inlet/outlet and the port. In various embodiments, the cooling and compression unit 40 is configured to provide the chilled fluid to the treatment cap assembly 10 and to receive the returning fluid from the treatment cap assembly 10, as will be described in greater detail below. In some embodiments, the fluid inlet 16 and the fluid outlet 18 may be embedded in the fluid circulating pad 12. The fluid inlet 16 and the fluid outlet 18 may be constructed of plastic and may be configured, for example, as quick connect fittings.

In various embodiments, the fluid circulating pad is constructed from two or more layers laminated together. The one or more fluid channels 20 can be formed within the fluid circulating pad 12 by welding or adhesively connecting various layers to one another. Alternatively, the one or more fluid channels 20 can be formed independently from the fluid circulating pad 12 (as opposed to being directly formed within the fluid circulating pad 12) by placing tubes or channels within layers of the fluid circulating pad 12.

The one or more fluid channels 20 can extend through the entire fluid circulating pad 12. Alternatively, separate fluid channels can be provided for various segments of the fluid circulating pad 12, with each fluid channel having an associated fluid inlet and fluid outlet. For example, a left half of the fluid circulating pad 12 can have a first fluid channel while a right half of the fluid circulating pad 12 can have a second fluid channel. As another example, the top half of the fluid circulating pad 12 can have a first fluid channel while a bottom half of the fluid circulating pad 12 can have a second fluid channel. Although fluid channels are described as being associated with particular “halves” of the fluid circulating pad 12, it is contemplated the fluid circulating pad 12 can be segmented into thirds, quarters, or into other discrete segments which can be equal or different in size. Moreover, it should be understood that fluid channels can be straight, zigzag, serpentine, or have any one of a number of patterns.

In various embodiments, the fluid circulating pad 12 is made of a light weight, flexible material that enables it to conform to the shape of the patient's head. In embodiments, at least a portion of the fluid circulating pad 12 is formed from a thermally conductive material such that the fluid circulating pad 12 enables heat transfer between the chilling fluid within the one or more fluid channels 20 and the skin in contact with the fluid circulating pad 12. The material selected can be fluid impermeable in embodiments. For example, a thin plastic, fabric, or foam may be used to construct all or part of the fluid circulating pad 12. In embodiments, the fluid circulating pad 12 can be made of a washable material.

In some embodiments, the fluid circulating pad 12 can include one or more sensors, including but not limited to temperature sensors and pressure sensors. When included, the sensors can be coupled to the cooling and compression unit 40 and are configured to collect and transmit information. For example, a temperature sensor can collect information regarding the temperature of the scalp and transmit it to the cooling and compression unit 40, which can regulate the fluid temperature or the flow rate of the chilling fluid based on the temperature collected by the sensor. Similarly, a pressure sensor can collect information regarding the pressure observed at the scalp as a result of the compression from the cold cap assembly and transmit it to the cooling and compression unit 40, which can regulate the pressure based on the observed pressure at the scalp. The sensors can be positioned at the fluid inlet 16 and fluid outlet 18, or at other points along the fluid circulating pad 12. In some embodiments, the sensors are removable from the fluid circulating pad 12.

In various embodiments, the treatment cap assembly 10 also includes a compression hood 114, as shown in FIGS. 3 and 4. The compression hood 114 is configured to receive compressed air or other gas from the cooling and compression unit 40 and to apply compression to the scalp of the patient. In embodiments, the compression hood 114 can be removably coupled to the fluid circulating pad 12, such as through hook and loop or other attachment mechanisms or fasteners. In various embodiments, the compression hood 114 includes two or more air bladders to enable the cooling and compression unit 40 to provide compressed gas or air in a sequential manner to the scalp of the patient. In embodiments, the compression hood 114 includes an embedded fitting that may be configured as a quick connect fitting. In some embodiments, the compression hood 114 includes a single fitting rather than an inlet and an outlet. Accordingly, in such embodiments, when the air is not pressurized, it may bleed through the fitting.

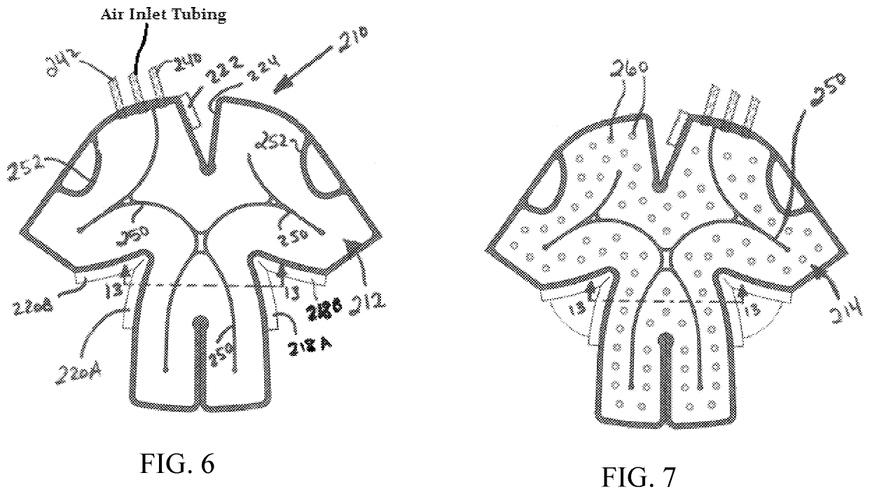

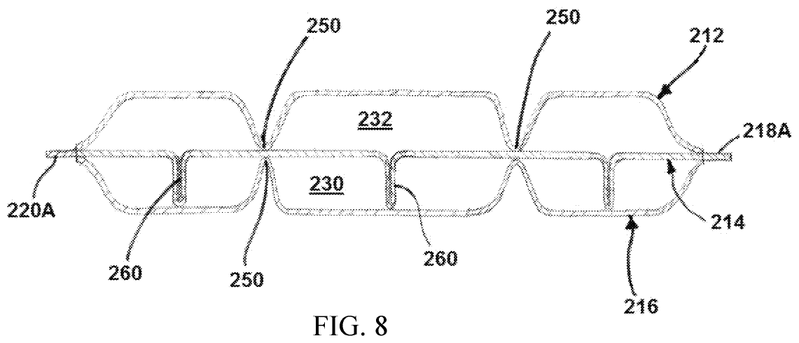

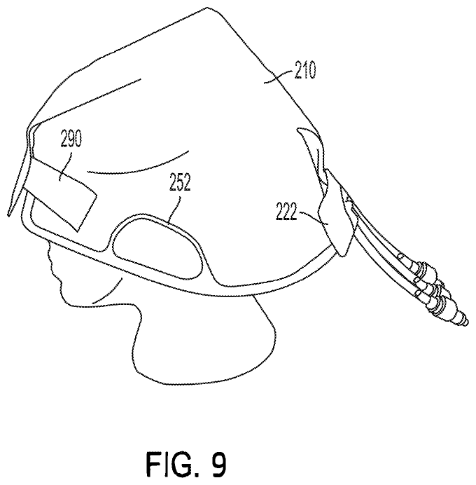

FIGS. 6-9 depict another example treatment cap assembly 210 for use in various embodiments. The treatment cap assembly 210 is configured to extend around the patient's scalp and conform thereto. The treatment cap assembly 210 includes at least three layers of material. In FIGS. 6 and 7, the treatment cap assembly 210 includes first layer 212, second layer 214, and third layer 216, which are sealingly attached at the periphery so as to form a cooling fluid chamber 230 and an air chamber 232 (shown in FIG. 8). Accordingly, cooling fluid is delivered to the cooling fluid chamber 230 through an inlet conduit 240, traverses through the cooling fluid chamber 230, and exits through an outlet conduit 242. As in other embodiments, the inlet conduit 240 and the outlet conduit 242 can be detachable from the fluid inlet and fluid outlet of the cooling and compression unit to receive the cooling fluid.

Similarly, compressed gas or air is delivered to the air chamber 232 through an inlet conduit. In some embodiments, the air chamber 232 can include more than one bladder (e.g., two or more bladders), as will be discussed in greater detail below, to enable sequential compression to be applied to the scalp during administration of chemotherapeutic agents. In embodiments, the cooling and compression unit 40 or the treatment cap assembly 210 can include a vent valve or other structure for controllably releasing air from the treatment cap assembly through the outlet such that air flows out of and into the outlet to control the air pressure within the cap.

The various layers of the treatment cap assembly 210 can be joined together by any known suitable method selected at least in part based on the materials selected for each of the layers. For example, when a plastic material is used, the layers 212, 214, and 216 can be joined together using heat sealing. In embodiments, the joining of the layers to one another defines the cooling fluid chamber 230 between the second layer 214 and the third layer 216, and the air chamber 232 between the first layer 212 and the second layer 214. Additional sealing can be provided between the various layers to form internal seams 250, 252, 260, such as to provide additional support in the form of support columns, define a serpentine or other suitable flow pattern within the cooling fluid chamber, to prevent the cooling fluid from passing to certain portions of the cooling fluid chamber 230, such as portions of the cooling fluid chamber 230 that will generally correspond to the location of a patient's ears, or to break up the flow of the chilling fluid and improve heat transfer.

Moreover, various tabs 218A, 218B, 220A, and 220B can be provided to assemble adjacent pieces together and to form a cap that has an overall shape that conforms to the skull of a patient. In embodiments, a fastener combination 222, 224 (e.g., a hook and loop fastener) can be provided to enable the diameter of the treatment cap assembly 210 to be adjusted for a proper fit. Other fasteners 290 can also be provided at one or more points along the treatment cap assembly 210 to enable further adjustment of the cap.

In some embodiments, the treatment cap includes an additional layer made of a biocompatible material, that is intended to be in contact with the patient's head. As used herein, a “biocompatible material” refers to any material that is compliant under ISO 10993-10 and ISO 10993-5.

It is contemplated that the treatment cap assembly can take other forms, as well.

### Cooling and Compression Units

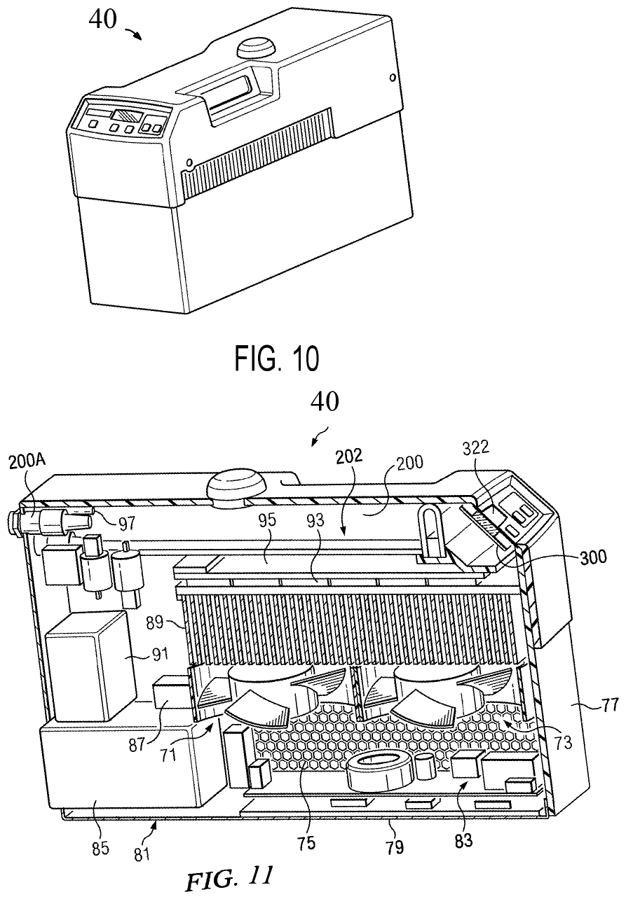

As described above, in various embodiments, the treatment cap assembly is coupled to a cooling and compression unit that is adapted to provide thermally controlled fluid and compressed gas for therapy. FIGS. 10-13 show a cooling and compression unit 40 that can be used with any of the treatment cap assemblies described hereinabove. Generally, the cooling and compression unit 40 includes a chassis having one or more perforations to allow for the low-pressure drawing of gas through the chassis to allow cooling of the components within the housing. The chassis provides protection to the components therein and enables the components to be easily transported between locations. In various embodiments, the chassis includes a handle, a display device, and one or more user inputs to enable users to provide input to the cooling and compression unit 40, as will be described.

As shown in FIG. 11, a heat transfer assembly 202 and a fluid reservoir 200 are shown. The fluid reservoir 200 is adapted for storage of liquid that can be pumped outwardly through a fluid outlet port 200A that is adapted for being coupled to the treatment cap assembly via connector tubes.

Also provided within the housing are fans 71 and 73, shown as being positioned above a grate 75. Grate 75 contains a filter portion 77. A lower portion of the grate 75 is connected to a bottom portion 79 of the chassis 81 to provide support for electronic components 83 mounted thereon. In embodiments, the electronic components 83 are configured to supply power to and to control the heat transfer assembly 202 and other elements within the cooling and compression unit 40. The fans 71 and 73 are positioned to push and/or pull gas from the grate 75 disposed peripherally about the electronic components 83 so that the flow of gas allows initial electronic cooling before being pushed into the top section of the chassis 81 where additional heat dissipation may be needed.

In FIG. 11, a power supply 85 is positioned in a lower portion of the chassis 81 and beneath a gas switch 87. The gas switch 87 is disposed beneath a heat sink 89 and adjacent to a fluid pump 91. In various embodiments, the power supply 85 can be a 500 Watt power supply. Additional power supplies can be utilized to power various components, depending on the particular embodiment. For example, in addition to the power supply 85, a 65 Watt power supply can be included to provide power for components requiring less power. In some embodiments, the power supplies are adapted to receive a plurality of inputs to enable the cooling and compression unit 40 to be utilized in conjunction with any one of a variety of electrical configurations.

The fluid pump 91 is shown positioned to collect fluid from the reservoir 200 that has been thermally controlled by the heat transfer assembly 202 for passage through the fluid outlet port 200A. Thermoelectric coolers (TECs) 93 are shown disposed between the heat sink 89 and a thermal transfer plate 95. Together, the TECs 93, the heat sink 89, and the thermal transfer plate 95 provide the requisite thermal control of the fluid within the reservoir 200. A gas connector 97 is shown disposed adjacent to the fluid outlet port 200A to provide the dissipation of gas for use in conjunction with the treatment cap assembly, for example, to apply pressure to force cooling fluid flowing from the fluid outlet port 200A to be in close contact with the patient.

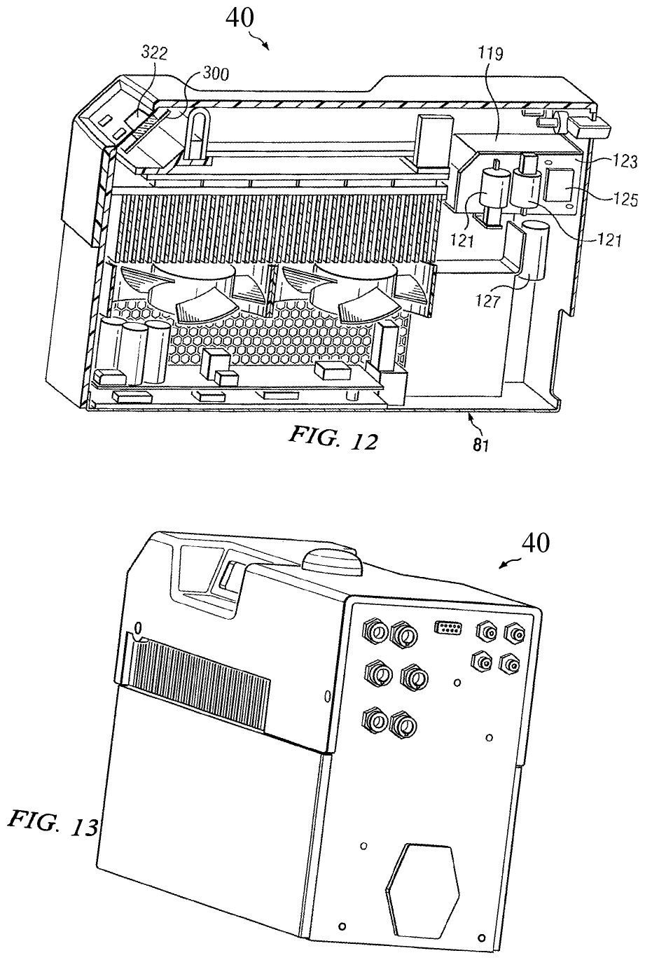

Referring now to FIG. 12, there is shown a cutaway perspective view of the cooling and compression unit 40 taken from an opposite side as is shown in FIG. 11. In conjunction with the cooling and compression therapy, a compression gas pump 119 is shown disposed adjacent to a plurality of solenoids 121 mounted to a compression gas bracket 123 adjacent the compression gas switch 125. In FIG. 12, an additional solenoid 127 is provided, although it should be understood that a greater or fewer number of solenoids and/or gas switches can be provided, depending on the particular implementation.

FIG. 13 illustrates a rear perspective view of the cooling and compression unit 40, in which the connectors and couplings on the rear panel are shown. In FIG. 13, a plurality of gas connectors and fluid connectors are included to provide thermally conditioned heat transfer fluid (e.g., cooling fluid) and to provide pressurized gas to one or more cooling and compression therapy devices (e.g., the treatment cap assembly). In embodiments, the fluid connectors are provided in plurality to facilitate circulation of fluid in a closed loop in an outward bound and an inward bound flow of fluid to and from the fluid reservoir for thermal control.

In various embodiments, a compression therapy device (e.g., the treatment cap assembly) is coupled to a plurality of gas connectors and the cooling and compression unit 40 is programmed to provide compressed gas in a dynamic manner to one or more bladders in the compression therapy device. In embodiments, the compression administered varies as a function of time. For example, compression can be applied for a period of time, followed by a period of rest during which compression is not applied. In one particular embodiment, compression is applied for a time period of from about 5 minutes to about 20 minutes, or from about 7.5 minutes to about 18 minutes, or from about 10 minutes to about 15 minutes, including any and all ranges and subranges therein. The period of rest can be from about 2 minutes to about 10 minutes, from about 3 minutes to about 7 minutes, or from about 4 minutes to about 6 minutes, including any and all ranges and subranges therein. In an example configuration, a compression cycle includes compression administration for about 15 minutes followed by a resting period of about 5 minutes, and the cycle is repeated during administration of the chemotherapeutic agents.

When compression is administered, in various embodiments, the amount of compression is sufficient to reduce or even eliminate blood flow to the hair follicles present on the scalp. Without being bound by theory, it is believed that reducing or eliminating blood flow to the hair follicles and reduce or even eliminate the exposure of the hair follicles to the chemotherapeutic agents administered to the individual and circulated through the blood stream, thereby reducing damage to the hair follicles and reducing hair loss that can result from chemotherapy treatments.

In some embodiments, compression is applied at a pressure of from about 0.5 mmHg to about 33 mmHg, from about 1 mmHg to about 33 mmHg, from about 5 mmHg to about 33 mmHg, from about 10 mmHg to about 33 mmHg, from about 0.5 mmHg to about 25 mmHg, from about 1 mmHg to about 25 mmHg, from about 5 mmHg to about 25 mmHg, or from about 10 mmHg to about 25 mmHg above ambient pressure, including any and all ranges and subranges therein. In some embodiments, the pressure applied at a pressure of from about 10 mmHg to about 30 mmHg, from about 12 mmHg to about 27 mmHg, or from about 13 mmHg to about 26 mmHg, relative to atmospheric pressure. In some embodiments, compression is applied at a level sufficient to reduce the blood flow to the follicles by an amount of about 10% or more, about 15% or more, about 20% or more, 25% or more, about 30% or more, about 35% or more, about 40% or more, about 45% or more, about 50% or more, about 55% or more, or about 60% or more, relative to an amount of blood flow to the follicles observed when compression is not being applied to the scalp. For example, compression can reduce the blood flow to the follicles from about 10% to about 80%, from about 10% to about 70%, from about 10% to about 60%, from about 10% to about 50%, from about 10% to about 40%, from about 10% to about 30%, from about 15% to about 80%, from about 15% to about 70%, from about 15% to about 60%, from about 15% to about 50%, from about 15% to about 40%, from about 15% to about 30%, from about 20% to about 80%, from about 20% to about 70%, from about 20% to about 60%, from about 10% to about 50%, from about 20% to about 40%, or from about 20% to about 30%, relative to the amount of blood flow to the follicles observed when compression is not being applied to the scalp, including any and all ranges and subranges therein. In some embodiments, compression can reduce the blood flow to the follicles from about 40% to about 80%, from about 40% to about 75%, or from about 40% to about 70%, relative to the amount of blood flow to the follicles observed when compression is not being applied to the scalp, including any and all ranges and subranges therein.

In some embodiments (e.g., in embodiments in which the treatment cap assembly includes two or more compression bladders), compressed gas can optionally be applied sequential manner. For example, a first bladder in the air chamber 232 (or the compression hood 114) can be inflated, followed by the inflation of a second bladder, which is followed by the inflation of a third bladder, and so on. The first bladder can be deflated before or after the second bladder is inflated, or the first bladder can remain inflated until all the bladders are inflated. In embodiments, the compression connectors are provided in plurality, although a single compression connector can be used for each compression therapy device. Alternatively, a plurality of compression connectors can be used by the compression therapy device. The compression pads are pressurized in accordance with the parameters set by the programming within the control boards of the cooling and compression unit 40.

FIG. 13 also illustrates an RS232 connector for data communication. However, it is contemplated that additional or alternative connections can be included, including but not limited to USB connections, wireless connections, or the like. The data communication connection can enable the input of patient data (e.g., patient name and/or other identifier, patient information such as weight, chemotherapeutic agents to be administered, chemotherapeutic dosage to be administered, duration of administration to chemotherapeutic agents, etc.) via electronic transfer. For example, a patient file can be loaded onto the cooling and compression unit 40 from a memory device (e.g., a portable USB memory device) or from a computer in electronic communication with the cooling and compression unit 40 and save the patient file in memory. In various embodiments, the control unit of the cooling and compression unit 40 is configured to use the inputted data to control the operation of the treatment cap assembly.

By way of example, based on the identity of the chemotherapeutic agents and dosages, and optionally the patient weight, duration of chemotherapeutic agent administration, etc., the control system can look up the half-life of the chemotherapeutic agents to be delivered in memory and calculate an appropriate duration of scalp cooling and compression. The display screen (FIG. 10) can then display instructions to the user, such as when to initiate therapy, provide an indication that the scalp has been sufficiently cooled to begin administration of the chemotherapeutic agents, and when scalp cooling and compression has ended. Accordingly, in embodiments, a library of data for various chemotherapeutic agents can be stored in memory, or can be accessed by the cooling and compression unit 40 through the data communication connection. Additionally or alternatively, various therapeutic protocols can be input and/or selected by a user (e.g., a medical practitioner).

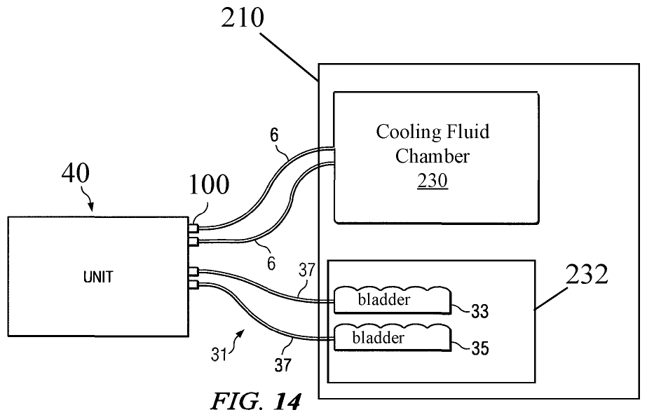

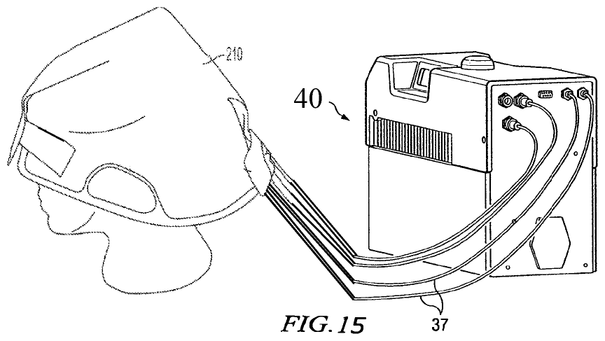

Turning now to FIG. 14, a cooling and compression control system for cooling and compression therapy is shown. The cooling and compression unit 40 is coupled to a treatment cap assembly 210 including a cooling fluid chamber 230 and an air chamber 232, as described above. The air chamber 232 includes a first bladder 33 and, optionally, a second bladder 35. The cooling fluid chamber 230 is coupled to the cooling and compression unit 40 by connector tubes 6 coupled to the cooling and compression unit 40 through a connector 100. The compression is provided through the first bladder 33 and the second bladder 35, which are coupled to the cooling and compression unit 40 through connector tubes 37. Any of the tubing can be, for example, polyethylene tubing conventionally known and used in medical devices. In various embodiments, the cooling and compression unit 40 generates a pulse to provide an amount of compressed gas to the first bladder 33 for a predetermined amount of time and at a predetermined pressure. In embodiments in which the air chamber includes a second bladder 35, the cooling and compression unit 40 generates a pulse to provide an amount of compressed gas to the second bladder 35. The pulses may be provided in rapid succession or a predetermined period of time can elapse between pulses. In embodiments, the pulse can be generated by opening a solenoid on compressed gas which provides an increased intensity of the pulse. In embodiments, a plurality of solenoids can be used, which can enable selection of the routing of the compressed gas.

As shown in FIG. 14, the connector tubes 37 are coupled to the cooling and compression unit 40 to provide pressurized gas in accordance with a pre-programmed application or user-specified parameters to maximize the effectiveness of the cooling and compression therapy. One activation technique is a high pressure, low ramp up sequence in which the selected pressure for alopecia reduction is provided without a high pulse rate. A high pulse rate time can be advantageous to modify the amount of blood flow to the patient's head and reduce follicle damage. Accordingly, the control boards of the cooling and compression unit 40 can provide a select pressurization in conjunction with the solenoids mounted within cooling and compression unit 40 (e.g., solenoids 121) to carefully control the pulse ramp time.

In various embodiments, different sequencing patterns, times, and pressures can be implemented. Various embodiments allow a plurality of parameters to be specified by a user, such as, for example, the inflated pressure, the deflated pressure, the rate of inflation, the inflation hold time, and the cycle time. For example, in one treatment sequence, the cooling and compression unit 40 can provide compressed gas to inflate bladder(s) of the air chamber for 3-20 seconds over a cycle time of a period of about 15 minutes or less, such that compression is administered for a period of about 15 minutes and followed by a period of about 5 minutes during which compression is not administered. As described hereinabove, the compression can be applied at a pressure of from about 0.5 mmHg to about 33 mmHg above ambient pressure, including any and all ranges and subranges therein. In some embodiments, deflation of one or more of the bladders of the air chamber can be a complete deflation, although in other embodiments, one or more bladders can remain partially compressed.

In FIG. 15, the cooling and compression unit 40 is shown connected to the treatment cap assembly 210 through three or more connector tubes 37. In the embodiment illustrated, two of the tubes deliver and return the cooling fluid and at least one other tube provides compressed gas for compression. In other embodiments, a different number of tubes can be connected and configured to provide cooling fluid or compressed gas, as described herein. For example, a first tube can provide compressed gas to a first bladder, a second tube can provide compressed gas to a second bladder, and so on.

In various embodiments, the cooling and compression unit 40 is configured to cool the treatment cap assembly 210 to a temperature of about 49° F. to about 55° F. at an ambient temperature of 77° F. in less than or equal to about 20 minutes and provide a compression of greater than or equal to approximately 0.5 mmHg above ambient pressure, including a compression of about 1 mmHg, about 5 mmHg, about 10 mmHg, about 15 mmHg, about 20 mmHg, about 25 mmHg, about 26 mmHg, about 27 mmHg, about 28 mmHg, about 29 mmHg, about 30 mmHg, about 31 mmHg, about 32 mmHg, or about 33 mmHg above ambient pressure, including any and all ranges and subranges therein. In some embodiments, the pressure within the treatment cap assembly 210 is maintained at a pressure of from about 10 mmHg to about 30 mmHg, from about 12 mmHg to about 27 mmHg, or from about 13 mmHg to about 26 mmHg, relative to atmospheric pressure. The connector 100 provides a fluid and/or gas connection between the cooling and compression unit 40 and the treatment cap assembly 210 for the transfer of gas and cooling fluid. The connector 100 may also allow for transfer of electrical sensor signals and/or data signals between the treatment cap assembly 210 and the cooling and compression unit 40.

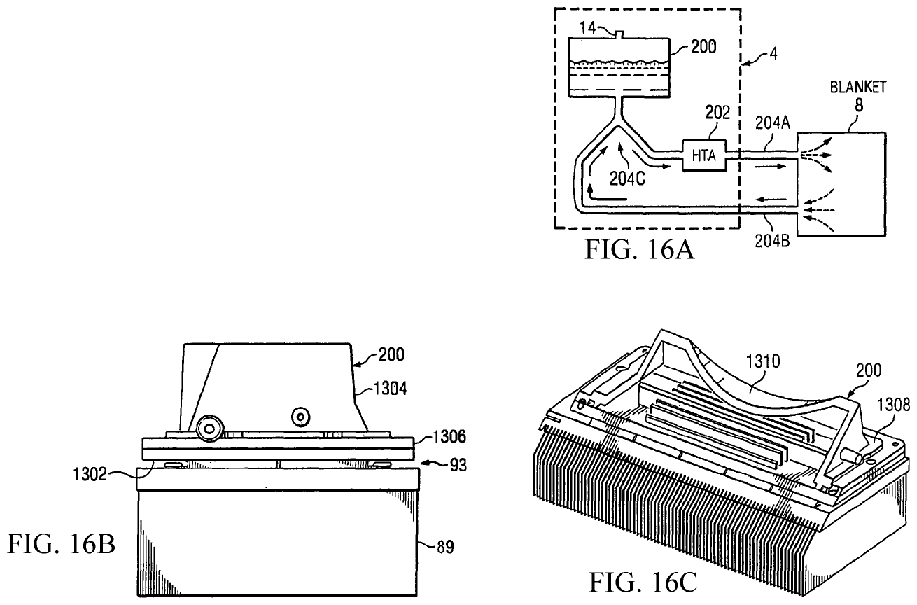

Referring now to FIG. 16A, a block diagram of one embodiment of the flow of cooling fluid between the cooling and compression unit 40 and the treatment cap assembly 210 is illustrated. The cooling and compression unit 40 includes a heat transfer fluid reservoir 200 and at least one heat transfer assembly 202 for heating and/or cooling the cooling fluid. Before the treatment cap assembly 210 is used for therapy, the system is primed with the cooling fluid. When the system is primed, substantially no gas exists in the tubes 204 between the reservoir 200, the heat transfer assembly 202, and the treatment cap assembly 210. In one embodiment, gas is actively and/or passively removed from the system to ensure that substantially no gas exists in the system. The flow tubes in the cooling and compression unit 40 between the reservoir 200, the heat transfer assembly 202, and the treatment cap assembly 210 form a three-point junction 204C. In one embodiment, the three-point junction 204C is formed as an inverted Y, however, other shapes and orientations are possible. By utilizing a three-point junction 204C, the cooling fluid returning from the treatment cap assembly 210 may be recirculated to the heat transfer assembly 202 without utilizing cooling fluid from the reservoir 200. The three-point junction 204C allows the heat transfer assembly 202 to cool the cooling fluid that has already been cooled prior to entering the treatment cap assembly 210. In one embodiment, the heat transfer assembly 202 does not cool the entire contents of the reservoir 200, but merely the portion of the cooling fluid that is currently circulating through the treatment cap assembly 210 and tubes 204. The reservoir 200 may be by-passed unless more fluid volume is needed. In the three-point junction 204C, cooling fluid returning from the treatment cap assembly 210 can be pumped, via a pump, to the heat transfer assembly 202. If more cooling fluid than that which is already circulating through the system is required, then the cooling fluid from the reservoir 200 may be introduced into the system. More fluid can be added via the opening 14, if needed.

In FIGS. 16B and 16C, the integration of the reservoir 200 and the heat transfer assembly 202 is illustrated. As shown in FIG. 16B, the rear of the reservoir 200 includes a coolant supply port 1302 for supplying cooling fluid to a fluid pump, a coolant return port 1304 for receiving cooling fluid from a treatment cap assembly 210, and a cold plate 1306. The cold plate 1306 can be positioned at the base of the reservoir 200 and is therefore in direct contact on its underside with the TEC 93. As shown in both FIGS. 16B and 16C, a divider 1308 is located in the middle of the reservoir 200 between the coolant supply port 1302 and the coolant return port 1304, thereby blocking direct flow of fluid between the two ports. As fluid flows into the back of the reservoir 200 through the coolant return port 1304, the divider 1308 channels the fluid to the front of the reservoir 200 and then back to the coolant supply port 1302. By preventing fluid from short circuiting directly from the coolant return port 1304 to the coolant supply port 1302, the divider 1308 forces exposure of the fluid to the cold plate 1306 which, as a result of its direct contact with the TEC 93, provides a surface are to cool the fluid. The reservoir 200 can also include vertical fins 1310 to further enhance contact areas with the fluid.

Other methods of cooling the cooling fluid can be employed, depending on the particular embodiment. For example, the cooling fluid may be circulated through a heat exchanger in functional contact with a chilled thermal mass. The mass can be any liquid, gas, or solid, such as dry ice, liquid nitrogen, non-polar solvents, and polar solvents. In one embodiments, the mass can be a water-based mass, either alone or in combination with another substance to adjust the freezing point to enable the mass to be cooled to a temperature below 0° C. Alternatively, the mass can be cooled to below freezing to permit the phase change of water from solid to liquid to absorb significantly more thermal energy from the circulating fluid than liquid water alone. The chilled thermal mass may be cooled internally within the cooling and compression unit 40 (e.g., by vapor compression) or can be added to an internal reservoir which is in thermal communication with the circulating cooling fluid (e.g., via a heat exchanger). In embodiments using a chilled thermal mass, scalp cooling can be controlled by regulating the flow rate of the cooling fluid through the cooling fluid pad. A circulating pump for pumping the cooling fluid may be thermally isolated from the chilled thermal mass, venting waste heat away from the unit and minimizing the amount of heat transferred to the chilled thermal mass and the cooling fluid.

As described above, one or more temperature sensors, mixing devices and other control schemes or devices permit the amount of thermal transfer, and thus the temperature of the cooling fluid, to be accurately controlled. For example, one or more temperature sensors can be included in or on the treatment cap assembly, such as in a location which ensures that the sensor will contact the patient's scalp and provide an accurate temperature signal to a control unit provided within the cooling and compression unit 40 and/or a temperature display on the cooling and compression unit 40. Accordingly, the temperature of the skin can be reduced in a controlled manner to a temperature of less than about 65° F., less than about 60° F., less than about 55° F., less than about 50° F., less than about 45° F., or even less than about 40° F. For example, the temperature of the skin can be reduced to a temperature of from about 35° F. to about 65° F., from about 35° F. to about 60° F., from about 35° F. to about 55° F., from about 35° F. to about 50° F., from about 35° F. to about 45° F., from about 35° F. to about 40° F., from about 40° F. to about 65° F., from about 40° F. to about 60° F., from about 40° F. to about 55° F., from about 40° F. to about 50° F., from about 40° F. to about 45° F., from about 45° F. to about 65° F., from about 45° F. to about 60° F., from about 45° F. to about 55° F., from about 45° F. to about 50° F., from about 50° F. to about 65° F., from about 50° F. to about 60° F., from about 50° F. to about 55° F., from about 55° F. to about 65° F., from about 55° F. to about 60° F., from about 60° F. to about 65° F., or from about 60° F. to about 62° F., including any and all ranges and subranges therein. It should be appreciated that if the temperature is too low, tissue damage or other injury may result. However, if the temperature is too high, efficacy of the treatment may result.

In various embodiments, the cooling and compression unit 40 is configured to provide precise temperature control as well as the duration of scalp cooling. Accordingly, the control unit within the cooling and compression unit 40 can be configured to provide a predetermined cooling regime. For example, the temperature of the cooling fluid can be gradually lowered from an ambient temperature to the desired cooling fluid temperature over a period of time (e.g., 30 minutes). Scalp temperature can be regulated by controlling the temperature of the cooling fluid and/or the flow rate of the cooling fluid through the pad. In embodiments, a timer function can be included to control a time-temperature profile for a patient. The profile can be customized for the patient and type of treatment. In addition, it can be programmed to discontinue cooling after a preset period of time or to provide cooling according to a timed loop (e.g., cool for 15 minutes, rest for 5 minutes, cool for 15 minutes, etc.).

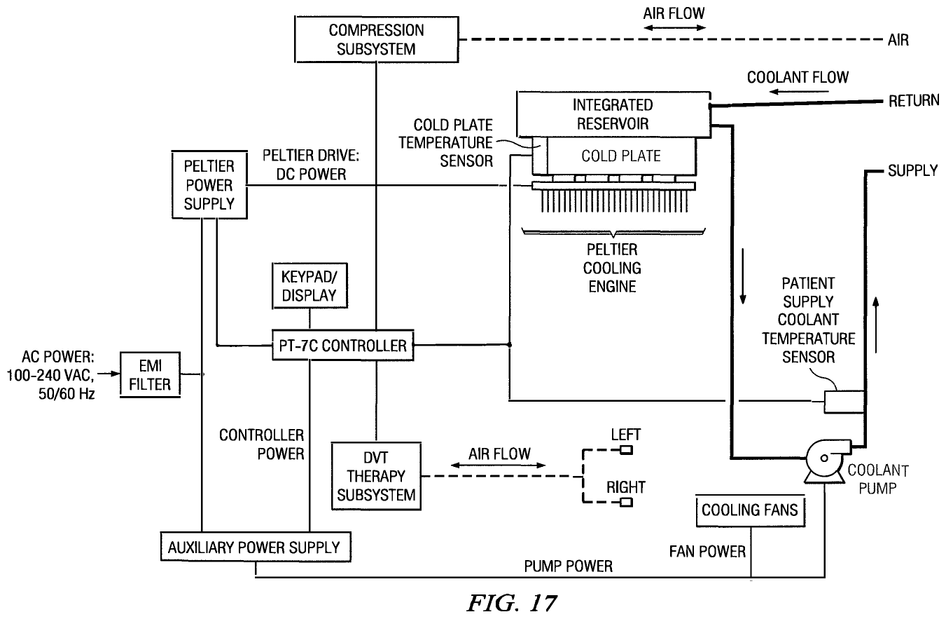

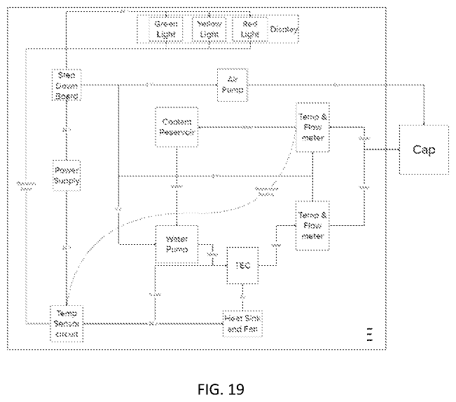

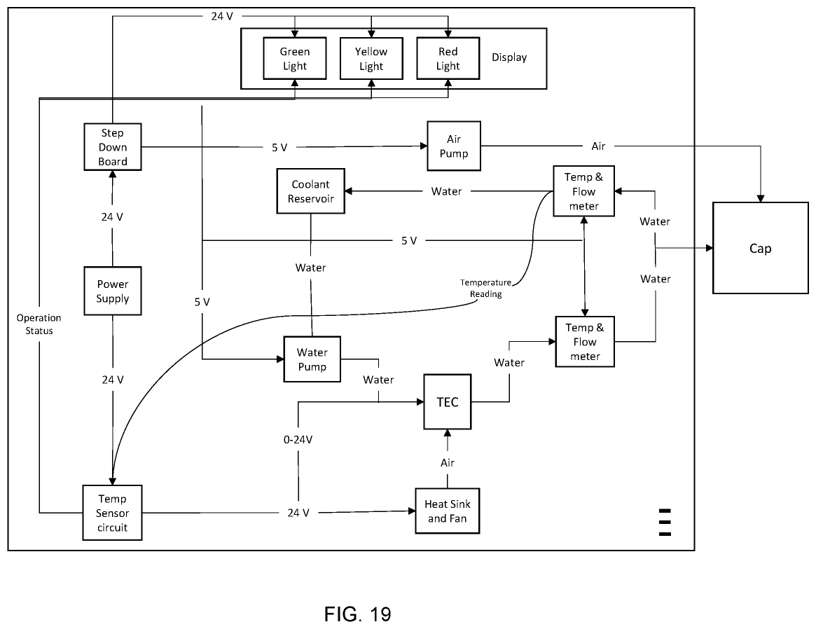

FIGS. 17 and 19 show example cooling and compression system block diagrams. In FIG. 17, gas is provided in a compression subsystem in conjunction with Peltier cooling of a fluid for cooling the scalp. In FIG. 17, the cooling fluid is thermally conditioned by the Peltier cooling engine, and cooling temperature sensors can be utilized. Coolant pumps are used in conjunction with cooling fans to provide selective cooling in an efficient manner for operation of the unit. The Peltier power supply is shown to be controlled by a PT-7C controller accessed via a keypad display. Various other features for control and power supply are also included, including an electro-magnetic interference (EMI) filter and auxiliary power supply used in conjunction with the compression subsystem. As shown in FIG. 17, the compression subsystem provides a separate gas flow for operation of at least two bladders for use in sequential compression treatment of a patient.

In FIG. 19, gas is provided in a compression subsystem via an air pump, and a TEC that is coupled to a heat sink and fan cools the water provided by a water pump before providing the water (i.e., cooling fluid) to the cap.

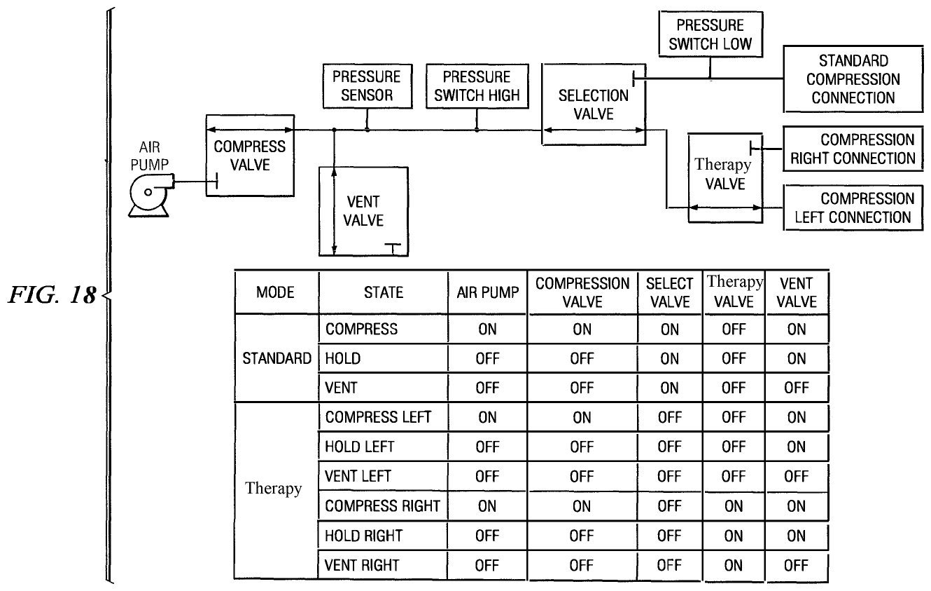

In FIG. 18, a compression therapy block diagram is illustrated. The gas pump is in flow communication with a compress valve which is utilized with a vent valve and pressure sensor. The pressure sensor is in association with a pressure switch high and pressure switch low. Various modes of operation utilizing the gas pump, compression valve, select valve, therapy valve, and vent valve are shown. In various embodiments, sequential compression can be utilized in combination with static compression.

In various embodiments, the cooling and compression unit 40 is configured to provide precise pressure control as well as the duration of scalp compression. Accordingly, the control unit within the cooling and compression unit 40 can be configured to provide a predetermined compression regime. In various embodiments, the cooling and compression unit 40 is configured to supply air at a controlled pressure of about 0.5 mmHg to about 33 mmHg, including any and all ranges and sub-ranges therein. Air pressure, duration of scalp compression, and other parameters of the pressurized air can be controlled according to a compression treatment profile for a patient. The profile can be customized for the patient and type of treatment. In addition, it can be programmed to discontinue compression after a preset period of time or to provide compression according to a timed loop (e.g., apply compression for 15 minutes, rest for 5 minutes, apply compression for 15 minutes, etc.).

In various embodiments, the treatment cap and cooling and compression unit are used to provide a prophylactic treatment for alopecia. In such treatments, the scalp of the individual is cooled to a temperature of less than about 65° F. and compression is periodically administered to the scalp during the administration of a dosage of a chemotherapeutic agent. In various embodiments, the combination of cooling and dynamic compression can reduce or eliminate alopecia as a result of the administration of the chemotherapeutic agents. In some embodiments, administering compression to the scalp of the individual is effective to reduce blood flow to follicles of the scalp from about 10% to about 50% or from about 20% to about 40% relative to an amount of blood flow to the follicles observed when compression is not administered to the scalp.

It will be appreciated that many more detailed aspects of the illustrated products and processes are in large measure, known in the art, and these aspects have been omitted for purposes of concisely presenting the general inventive concepts. Although the present invention has been described with reference to particular means, materials and embodiments, from the foregoing description, one skilled in the art can easily ascertain the essential characteristics of the present disclosure and various changes and modifications can be made to adapt the various uses and characteristics without departing from the spirit and scope of the present invention as described above and set forth in the attached claims.

What is claimed is:

1. A method comprising:
administering to an individual a dosage of a chemotherapeutic agent;
cooling a scalp of the individual to a temperature of less than about 65° F.; and
periodically administering compression to the scalp of the individual during the step of administering the dosage of the chemotherapeutic agent.

2. The method according to claim 1, wherein periodically administering compression to the scalp comprises:
administering compression for a time period of from about 5 minutes to about 20 minutes;
a period of rest of from about 2 minutes to about 10 minutes during which compression is not administered; and
repeating the administering compression and the period of rest.

3. The method according to claim 2, wherein the time period for administering compression is from about 7.5 minutes to about 18 minutes.

4. The method according to claim 2, wherein the period of rest is from about 3 minutes to about 7 minutes.

5. The method according to claim 1, wherein the compression is administered at a pressure of from about 0.5 mmHg to about 33 mmHg above ambient pressure.

6. The method according to claim 1, wherein cooling the scalp of the individual comprises cooling the scalp of the individual to a temperature of from about 60° F. to about 62° F.

7. The method according to claim 1, wherein administering compression to the scalp of the individual is effective to reduce blood flow to follicles of the scalp from about 10% to about 50% relative to an amount of blood flow to the follicles observed when compression is not administered to the scalp.

8. The method according to claim 1, wherein the cooling the scalp begins prior to the administering to the individual the dosage of the chemotherapeutic agent.

9. The method according to claim 1, wherein the cooling the scalp continues following completion of the administering to the individual the dosage of the chemotherapeutic agent.

10. The method according to claim 1, wherein the periodically administering compression begins prior to the administering to the individual the dosage of the chemotherapeutic agent.

11. The method according to claim 1, wherein the periodically administering compression continues following completion of the administering to the individual the dosage of the chemotherapeutic agent.

12. A system comprising:
a treatment cap assembly comprising:
a fluid circulating pad configured to circulate a cooling fluid; and
a compression hood configured to administer compression to a scalp of an individual wearing the treatment cap assembly; and
a cooling and compression unit coupled to the treatment cap assembly configured to provide the cooling fluid to the fluid circulating pad and to periodically provide compressed gas to the compression hood.

13. The system according to claim 12, wherein the fluid circulating pad comprises:
one or more fluid channels through which the cooling fluid is circulated;
a fluid inlet in fluid communication with the cooling and compression unit to receive cooling fluid from the cooling and compression unit; and
a fluid outlet in fluid communication with the cooling and compression unit to provide the cooling fluid to the cooling and compression unit after circulation through the one or more fluid channels.

14. The system of claim 12, wherein the fluid circulating pad includes one or more sensors communicatively coupled to the cooling and compression unit and configured to collect and transmit information to the cooling and compression unit.

15. The system of claim 12, wherein the compression hood is removably coupled to the fluid circulating pad.

16. The system of claim 12, wherein the compression hood comprises:
an inlet conduit fluidly coupled to the cooling and compression unit to receive compressed gas from the cooling and compression unit; and
a vent configured to controllably release air from the compression hood.

17. The system according to claim 12, wherein the cooling and compression unit is configured to:
provide compressed gas to the compression hood for a time period of from about 5 minutes to about 20 minutes;
cease providing compressed gas to the compression hood for a period of rest of from about 2 minutes to about 10 minutes; and
repeat the providing compressed gas and the period of rest.

18. The system according to claim 12, wherein the compression is administered at a pressure of from about 0.5 mmHg to about 33 mmHg above ambient pressure.

19. The system according to claim 12, wherein the circulating cooling fluid is effective to cool the scalp of the individual to a temperature of less than about 65° F.

20. The system according to claim 12, wherein administering compression to the scalp of the individual is effective to reduce blood flow to follicles of the scalp from about 10% to about 50% relative to an amount of blood flow to the follicles observed when compression is not administered to the scalp.
